(19) United States
(12) Patent Application Publication
(10) Pub. No.: US 2019/0269831 A1
(43) Pub. Date: Sep. 5, 2019

(54) COATING FOR MEDICAL EQUIPMENT AND ENDOSCOPE

(71) Applicant: OLYMPUS CORPORATION, Tokyo (JP)

(72) Inventors: Minoru HARA, Yokohama-shi (JP); Takashi MAGARA, Tokyo (JP)

(73) Assignee: OLYMPUS CORPORATION, Tokyo (JP)

(21) Appl. No.: 16/416,689

(22) Filed: May 20, 2019

Related U.S. Application Data
(63) Continuation of application No. PCT/JP2018/004170, filed on Feb. 7, 2018.

(30) Foreign Application Priority Data
Feb. 15, 2017 (JP) 2017-025984

(51) Int. Cl. A61L 29/08; A61B 1/005; A61L 29/14; C09D 175/04

(52) U.S. Cl. CPC A61L 29/08; A61B 1/005; C09D 7/40; C09D 175/04; A61L 29/14

(57) ABSTRACT

A coating for medical equipment includes: an isocyanate-curable coating composition; and a radical scavenger.

### CROSS REFERENCE TO RELATED APPLICATIONS

This application is a continuation application based on a PCT Patent Application No. PCT/JP2018/004170, filed on Feb. 7, 2018, whose priority is claimed on Japanese Patent Application No. 2017-025984, filed Feb. 15, 2017. The contents of both the PCT Application and the Japanese Application are incorporated herein by reference.

### BACKGROUND

### Technical Field

The present invention relates to a coating for medical equipment and an endoscope.

### Background Art

Conventionally, for example, in medical equipment such as endoscopes and treatment tools, various indicators including marks and characters may be applied or printed on a site to be inserted into a patient's body using a coating for a medical equipment.

The medical equipment may touch a drug solution or be heated up for example, during sterilization. For this reason, coatings used for an indicator on medical equipment need to have chemical resistance and heat resistance after curing. Furthermore, in many cases, the site on medical equipment to which the coating material for medical equipment is applied curves at the time of insertion in the body. For this reason, coatings for medical equipment are required to have flexibility to withstand bending after curing.

For example, Japanese Patent (Granted) Publication No. 3776783 describes an endoscope indicator composition including a binder comprising a fluorine-containing copolymer and a non-yellowing isocyanate-based curing agent.

It is described that the indicator composition of the endoscope disclosed in Japanese Patent (Granted) Publication No. 3776783 has improved resistance to sterilization using hydrogen peroxide and low temperature plasma in combination.

However, since the cured product obtained by curing the medical equipment coating is formed to be in close contact with the surface of the medical equipment body, it is susceptible to low temperature plasma. Therefore, the resistance to low temperature plasma sterilization is often inferior to that of the medical equipment body. In particular, when low-temperature plasma sterilization is repeated, the cured product may peel off from the surface of the medical equipment main body, resulting in a problem that the product life of the medical equipment main body is shortened.

For this reason, it is strongly demanded to further improve the resistance to the low temperature plasma sterilization of the coating for medical equipment applied to the medical equipment main body from the viewpoint of suppression of medical expenses and the like.

### SUMMARY

A coating for medical equipment includes: an isocyanate-curable coating composition; and a radical scavenger.

In the coating for medical equipment, the radical scavenger may include at least one of hydroquinone and benzoquinone.

In the coating for medical equipment, the isocyanate-curable coating composition may include a fluorine-type compound which introduces a fluoro group into a hardened material after a polymerization reaction.

In the coating for medical equipment, the isocyanate-curable coating composition may include a main agent and a curing agent for polymerizing the main agent.

An endoscope includes a coating layer formed of a coating for medical equipment, the coating including: an isocyanate-curable coating composition; and a radical scavenger.

The endoscope may include an insertion portion inserted into a patient's body, the insertion portion may include a flexible tubular portion formed in a flexible tube shape, the flexible tube portion may include a flexible tube and an outer-layer resin covering an outer peripheral portion of the flexible tube in a tubular shape, and the coating for medical equipment may be applied to a surface of the outer-layer resin.

According to the coating for medical equipment and the endoscope of the present invention, the durability against low temperature plasma sterilization can be improved.

### BRIEF DESCRIPTION OF THE DRAWINGS

FIG. 1 is a schematic perspective view showing a configuration example of a medical equipment according to an embodiment of the present invention.

FIG. 2 is a schematic cross-sectional view showing the configuration of a coating film layer in the medical equipment of the embodiment of the present invention.

### DETAILED DESCRIPTION OF THE PREFERRED EMBODIMENTS

Hereinafter, a medical equipment and a coating for a medical equipment according to an embodiment of the present invention will be described.

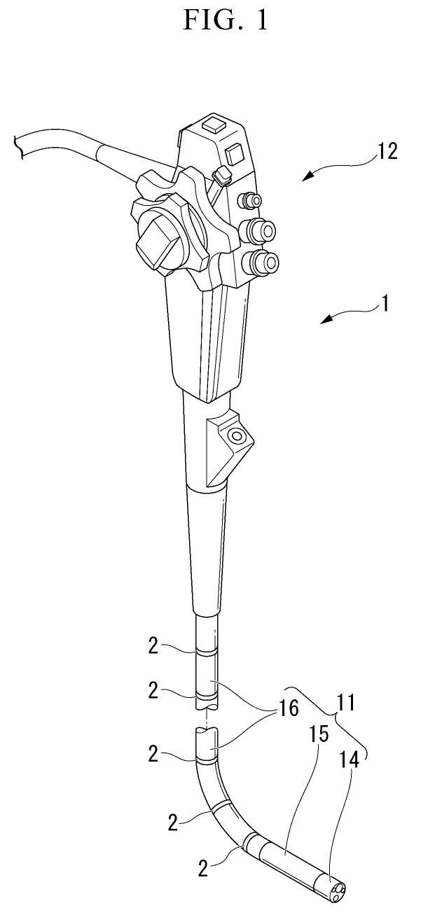

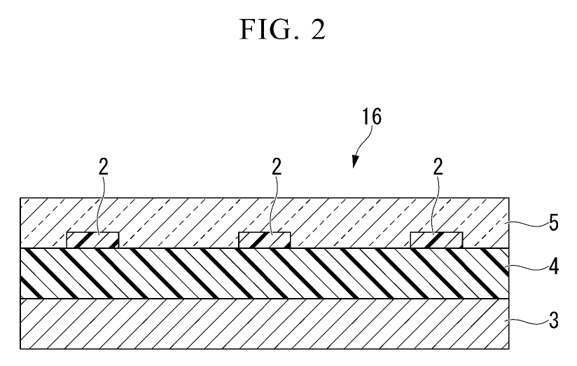

FIG. 1 is a schematic perspective view showing a configuration example of a medical equipment according to an embodiment of the present invention. FIG. 2 is a schematic cross-sectional view showing the configuration of a coating layer in the medical equipment of the embodiment of the present invention.

As shown in FIG. 1, the endoscope 1 (medical equipment) of the present embodiment includes an insertion portion 11 and an operation unit 12.

The insertion portion 11 is formed in a flexible tubular shape for insertion into a patient's body. The insertion portion 11 is provided with a distal end portion 14, a bending portion 15, and a flexible tube portion 16 in order from the distal end side in the insertion direction. Although not particularly shown, a treatment instrument channel for passing a treatment instrument may be provided along the longitudinal direction inside the insertion portion 11.

The distal end portion 14 is a portion which is disposed at the distal end portion of the endoscope 1 and includes an end effector as a manipulator. In the present embodiment, in order to acquire an image of a subject, the distal end portion 14 includes an imaging element such as a CCD and an imaging optical system including an appropriate lens inside, and has a cylindrical outer shape.

An imaging window and an illumination window are formed at the distal end of the distal end portion 14. When the insertion portion 11 includes a treatment instrument channel, an opening of the treatment instrument channel is provided at the distal end of the distal end portion 14.

The bending portion 15 is connected to the proximal end side of the distal end portion 14. The bending portion 15 is a tubular portion that can be bent in order to change the direction of the distal end portion 14.

In the bending portion 15, for example, a plurality of annular node rings are movably connected, and a plurality of angle wires are inserted therein.

In the inside of the bending portion 15, for example, members such as an electric wiring connected to the imaging element of the distal end portion 14 and a light guide extended to the illumination window are accommodated. The members such as the electrical wiring and the light guide are inserted into the inside of the flexible tube portion 16 described later and run to the operation unit 12 described later.

The flexible tube portion 16 is a tubular portion connecting the bending portion 15 and the operation unit 12 described later.

The flexible tube portion 16 includes, for example, a flexible tube in which a belt-like member made of metal or resin is spirally wound, and a soft outer-layer resin. The outer-layer resin covers the outer peripheral portion of the flexible tube in a tubular manner.

For example, one or more resins selected from styrene resins, olefin resins, vinyl chloride resins, polyester resins, polyurethane resins, and nylon resins may be used as the material of the outer-layer resin.

With such a configuration, the flexible tube portion 16 can be bent in an appropriate direction while holding a substantially circular cross section.

Inside the flexible tube portion 16, each angle wire extended from the bending portion 15 to the proximal end side is inserted into a coil sheath disposed in the flexible tube portion 16. Similar to the bending portion 15, members such as the above-mentioned electrical wiring and light guide are inserted into the inside of the flexible tube portion 16.

In the flexible tube portion 16, an indicator or index or marker 2 (coating layer) visible from the outside is formed. The indicator 2 is a mark provided so that the operator can easily understand the length of the insertion portion 11 inserted into the patient's body.

The formation position, the shape, and the number of the indicators 2 are not particularly limited. In the present embodiment, as an example, annular marks that encircle the outer peripheral portion of the flexible tube portion 16 are disposed at equal intervals in the longitudinal direction of the flexible tube portion 16. Although not shown in FIG. 1, as the indicator 2, along with such an annular mark, numerals, letters, symbols, or the like representing the length from the distal end portion may be drawn.

In FIG. 2, an example of sectional drawing of the site in which the indicator 2 was formed on the flexible tube portion 16 is shown.

As shown in FIG. 2, the indicator 2 is formed on the surface of the outer-layer resin 4 that covers the flexible tube 3.

The indicator 2 is formed of a coating film layer made of a cured product of the coating material for medical equipment of the present embodiment.

In the example shown in FIG. 2, the indicator 2 and the outer-layer resin 4 are coated by the coat layer 5.

The coat layer 5 is a resin layer that protects the indicator 2 and the outer-layer resin 4. In the present embodiment, the coat layer 5 is formed over the entire length of the flexible tube portion 16.

As a resin material of the coat layer 5, an appropriate resin material which is excellent in flexibility and can be safely inserted into a living body may be used. More preferably, the resin material of the coat layer 5 has chemical resistance.

The coat layer 5 may be a single layer coat or a multilayer coat. In the case of the present embodiment, the coat layer 5 uses a transparent material at least in the range covering the indicator 2.

For example, as a resin material of the coat layer 5, a urethane resin (urethane resin composition) may be used. Since the urethane resin is excellent in flexibility, it is particularly suitable as a material of the coat layer 5 covering the outer-layer resin 4 of the insertion portion 11.

Among the urethane-based resins, a resin material particularly suitable for the coating layer 5 is a fluorine-based urethane resin (urethane-based resin composition) which is excellent in chemical resistance.

As shown in FIG. 1, the operation unit 12 is a device portion where the operator operates the endoscope 1. As an example of the operation performed through the operation unit 12, an operation of pulling an angle wire to change the amount of bending of the bending portion 15 can be picked up. The operation unit 12 includes an operation knob, an operation switch, and the like.

Next, the medical equipment coating of the present embodiment for forming the indicator 2 will be described.

The coating for medical equipment of the present embodiment is configured to include an isocyanate-curable coating composition and a radical scavenger.

An isocyanate-curable coating composition has a composition capable of producing a cured resin through a curing reaction with an isocyanate group. The type of a resin cured product of the isocyanate-curable coating composition and the constitution of a main skeleton are not particularly limited as long as they are produced by polymerization reaction of an isocyanate group.

For example, the isocyanate-curable coating composition may be a urethane-based coating material containing a polyol as a main ingredient. The polyol undergoes a polymerization reaction with an isocyanate-based curing agent (curing agent) having an isocyanate group to form a urethane bond. The number of hydroxy groups in the polyol and the type of main skeleton are not particularly limited.

The isocyanate-curable coating composition can form a urethane resin cured product when a polyol is contained as a main ingredient.

Examples of specific polyols include, for example, fluorinated polyols, acrylic modified polyols, polyester polyols, polyether polyols, epoxy polyols, and polyolefin polyols.

An isocyanate-based curing agent may be prepared separately from the isocyanate-curable coating composition. In this case, the combination of the isocyanate-curable coating composition and the isocyanate-based curing agent constitutes a two-component curable coating set.

The isocyanate-curable coating composition is cured by mixing an isocyanate-based curing agent. In this case, in order to shorten the curing time, the mixture of the isocyanate-curable coating composition and the isocyanate-based curing agent may be heated, if necessary.

The isocyanate-based curing agent may be premixed in the isocyanate-curable coating composition. In this case, the isocyanate-curable coating composition constitutes a one-component curable coating consisting of a mixture of the main ingredient and the isocyanate-based curing agent.

The curing agent for curing the isocyanate-curable coating composition is not limited to the above-mentioned isocyanate-based curing agent. For example, when the main agent contains an isocyanate group, an appropriate compound having a functional group that causes a polymerization reaction with the isocyanate group of the main agent may be used as the curing agent.

A curing agent that causes a main agent containing an isocyanate group to undergo a polymerization reaction may constitute a two-component curable coating set. Alternatively, a curing agent that causes a main agent containing an isocyanate group to undergo a polymerization reaction may constitute a one-part curable coating.

The main component of the isocyanate-curable coating composition may contain a fluorine-based compound for introducing a fluoro group into the cured product through the polymerization reaction. Here, “a fluorine-based compound that introduces a fluoro group into a cured product through polymerization reaction” means a fluorine-based compound in which a fluoro group in the fluorine-based compound or a skeleton containing a fluoro group is contained into a polymer after the polymerization reaction.

The fluoro group is preferably introduced into the main chain of the polymer in the cured product. For example, the main agent may include a fluorinated polyol.

The curing agent for curing the isocyanate-curable coating composition may contain a fluorine-based compound for introducing a fluoro group into the cured product through the polymerization reaction. The fluoro group is more preferably introduced into the main chain of the polymer in the cured product. For example, the curing agent may contain a fluorine-based isocyanate compound.

As the radical scavenger, an appropriate compound which can react with radicals generated in plasma to trap the radicals can be used. Examples of radical scavengers include, for example, hydroquinone and benzoquinone.

However, as the radical scavenger, for example, a hydroquinone compound composed of a derivative of hydroquinone or a benzoquinone compound composed of a derivative of benzoquinone may be used.

Hydroquinone (benzoquinone) is preferable as a radical scavenger because it has a low molecular weight compared to other hydroquinone compounds (benzoquinone compounds).

Hydroquinones, hydroquinone compounds, benzoquinones and benzoquinone compounds may be used as polymerization inhibitors. However, in the polymerization by isocyanate curing, hydroquinone, hydroquinone compounds, benzoquinone and benzoquinone compounds do not act as polymerization inhibitors.

Examples of other radical scavengers include, for example, butyl catechol, butyl hydroxytoluene, hydroquinone monomethyl ether, phenothiazine and the like.

The coating for medical equipment may contain additives other than the radical scavenger if necessary. Examples of such additives include rubber materials, solvents, coloring materials and the like.

As a rubber material, an appropriate material for improving the flexibility of a cured product of a coating for a medical equipment is used.

Examples of the rubber material include, for example, liquid polyisoprene, liquid polybutadiene, liquid acrylonitrile-butadiene rubber, liquid polychloroprene, liquid polyoxypropylene, liquid polyoxytetramethylene glycol, liquid polyolefin glycol, liquid poly-ε-caprolactone, liquid Polysulfide rubber, liquid fluoro rubber, liquid polyisobutylene and the like.

The solvent may be contained in an appropriate amount in the medical equipment coating so that the medical equipment coating can be easily applied. As the solvent, an appropriate organic solvent or a mixed solution of the organic solvent is used as needed.

As the coloring material contained in the coating for medical equipment, an appropriate pigment or the like having a necessary color is used according to the application of the coating for medical equipment. Since the endoscope 1 in which the coating for a medical equipment is used is sterilized, a material having heat resistance that can withstand at least the sterilization temperature is used as the material of the coloring material.

As a pigment used for a coating for medical equipment, for example, pigments of single colors such as white, red, yellow, green, blue and black, or pigments in which two or more kinds of pigments of these single colors are mixed can be used. As a coloring material, a dye may be used.

Examples of materials suitable for the colorant include, for example, titanium oxide (titanium white), carbon black, chromium yellow and the like. In particular, titanium oxide is likely to shield ultraviolet rays, so that it is possible to improve the durability of the coating for medical equipment against the sterilization method in which ultraviolet rays are generated, as in the low temperature plasma sterilization method.

Next, a method of forming the indicator 2 will be described.

First, the coating material for a medical equipment described above is applied to an object member of the indicator 2 in a state of containing a curing agent. For example, if the isocyanate-curable coating composition does not contain a curing agent, the curing agent is mixed at any time before application.

The application method of the coating for medical equipment is not particularly limited. Examples of the method of applying the coating material for a medical equipment include screen printing, offset printing, inkjet printing, and the like.

The application area of the indicator 2 is an area necessitated to form the shape of the indicator 2. In the example shown in FIG. 2, the application object of the indicator 2 is the outer-layer resin 4 in which the flexible tube 3 is inserted.

Thereafter, heating is performed to cure the applied coating for medical equipment. The heating temperature is a temperature at which the polymerization reaction of the isocyanate-curable coating composition proceeds in the coating for a medical equipment.

Thus, when the coating for medical equipment is heated, the polymerization of the isocyanate-curable coating composition proceeds, and the coating for medical equipment is cured. The cured product of the medical equipment coating constitutes the indicator 2.

Thereafter, a coating material for forming the coating layer 5 is applied so as to cover the indicator 2 and the outer-layer resin 4. Thereafter, a curing process is performed to cure the coating material.

Thus, a laminated structure of the outer-layer resin 4, the indicator 2, and the coat layer 5 as shown in FIG. 2 is formed.

The action of the coating for medical equipment of the present embodiment and the cured product thereof will be described.

The indicator 2 which is a cured product of the coating for a medical equipment of the present embodiment is particularly exposed to a sterilizing gas, a sterilizing agent and the like when it is sterilized after use. In particular, for example, low temperature plasma sterilization of a peroxide gas system using hydrogen peroxide is used for sterilization of a medical equipment.

In low temperature plasma sterilization, an object to be sterilized is exposed to a sterilizing gas that forms a low temperature plasma. When the sterile gas acts on the bacteria, the sterile gas kills the bacteria. However, low temperature plasma may also act on the polymer on the surface of the medical equipment, for example, to cut the polymer polymerization structure and the like. When the chemical bond of the polymer is broken, the cured product containing the polymer is weakened. Specifically, cracking or peeling of a cured product containing a polymer may occur.

The present inventors have made earnest studies, particularly considering that radical attack in a low temperature plasma contributes to polymer cleavage. The present inventors have found that the resistance to low-temperature plasma sterilization is improved by adding a radical scavenger used for a polymerization inhibitor or the like to a coating for a medical equipment, and have reached to the present invention.

The indicator 2 of the present embodiment is formed by curing a medical equipment coating containing a radical scavenger. Since the radical scavenger is not consumed in the curing reaction concerning the isocyanate group when curing the coating for a medical equipment, the indicator 2 contains the radical scavenger.

The radical scavenger traps radicals by reacting with radicals, so that radicals acting on the polymer which is a cured product of the main agent contained in the indicator 2 can be reduced. Therefore, the radical scavenger can suppress the weakness of the indicator 2 due to the reaction with the radical.

When a radical acts on a functional group that contributes to chemical bonding or adhesion at the interface of the indicator 2 and the outer-layer resin 4 or at the interface of the indicator 2 and the coating layer 5, the chemical bond or functional group may be damaged. In this case, the adhesion at each interface is reduced.

However, since the indicator 2 contains a radical scavenger, the action of such radicals is also suppressed. The radical scavenger can also suppress the weakening in the adhesion between the indicator 2 and the outer-layer resin 4 and the adhesion between the indicator 2 and the coat layer 5.

As described above, the durability of the indicator 2 in low temperature plasma sterilization is improved as compared to the case where the radical scavenger is not contained. Since the durability of the indicator 2 is improved, the durability of the endoscope 1 is also improved.

Radicals trapped by the radical scavenger are not limited to radicals generated in low temperature plasma sterilization. For this reason, the indicator 2 has the same function also in the case where it receives radical attack other than at low temperature plasma sterilization, for example, when it receives radical attack at the time of use, storage and the like of a medical equipment.

When the coating for medical equipment contains at least one of hydroquinone and benzoquinone which are low molecular weight radical scavengers, the amount of radicals trapped per the scavenger's unit mass is large. Therefore, when at least one of hydroquinone and benzoquinone is used as the radical scavenger, the durability of indicator 2 can be efficiently improved even with a small addition amount.

When the isocyanate-curable coating composition of the coating for medical equipment or the curing agent contains a fluorine-based compound for introducing a fluoro group into the cured product after the polymerization reaction, the fluoro group is introduced into the cured product of the coating for medical equipment. Since the fluorine-based resin containing a fluoro group tends to be negatively charged, it is resistant to the attack of radicals generated by sterilization. As a result, the cured coating containing a fluoro group has superior sterilization resistance as compared to a cured coating containing no fluoro group.

In the description of the above embodiment, an example in which the medical equipment is the endoscope 1 has been described. However, the medical equipment which can use the coating for medical equipment of the present invention is not limited to an endoscope. The medical equipment coating of the present invention may be used, for example, in medical equipment such as treatment tools, catheters, stents, syringes and surgical energy treatment devices.

In the description of the above embodiment, the example in the case where the coating film layer formed on the medical equipment is the indicator 2 has been described. However, the coating film layer formed on the medical equipment by the medical equipment coating of the present invention is not limited to the indicator 2. The coating film layer formed on the medical equipment by the coating material for medical equipment of the present invention may be, for example, a coating film layer that describes characters, symbols, patterns, or the like that do not have a function as an indicator. The coating layer formed on the medical equipment by the coating material for a medical equipment of the present invention may be, for example, a functional layer such as a protective film layer for protecting the surface of the medical equipment or a low friction layer for reducing the friction of the surface of the medical equipment.

In the description of the above embodiment, the example in which the coating for a medical equipment contains a coloring material has been described. However, the coloring material may not be included when, for example, it may be transparent as in the case of using for applications other than the indicator.

In the description of the above embodiment, the example in which the indicator 2 and the outer-layer resin 4 are coated with the coat layer 5 forming the outermost layer of the flexible tube portion 16 has been described, but the coat layer 5 may not be the outermost layer of the flexible tube portion 16 as long as the coat layer 5 is laminated on the indicator 2.

Furthermore, when it is not necessary to provide a protective layer on at least one of the indicator 2 and the outer-layer resin 4, the coat layer 5 may be omitted at a site where the protective layer may not be provided.

### Example

Next, Examples 1 to 3 of the coating for a medical equipment of the above-described embodiment will be described together with Comparative Example 1. The coating composition and evaluation result of each example and comparative example are shown in the following Table 1.

TABLE 1

| | PAINT COMPOSITION | | | | | | | | |
|---|---|---|---|---|---|---|---|---|---|
| | MAIN INGREDIENT | | RADICAL SCAVENGER | | HARDENING AGENT | | COLOR MATERIAL | | |
| | | PARTS | | PARTS | | PARTS | | PARTS | EVALUATION |
| | MATERIAL | BY | MATERIAL | BY | MATERIAL | BY | MATERIAL | BY | RESULTS |
| | NAME | MASS | NAME | MASS | NAME | MASS | NAME | MASS | ADHESION |
| EXAMPLE 1 | FLUORINATED | 100 | HYDROQUINONE | 5 | ISOCYANATE | 24 | TITANIUM | 20 | 0 |
| | POLYOL | | | | | | OXIDE | | |
| EXAMPLE 2 | FLUORINATED | 100 | BENZOQUINONE | 5 | ISOCYANATE | 24 | TITANIUM | 20 | 0 |
| | POLYOL | | | | | | OXIDE | | |
| EXAMPLE 3 | EPOXY | 20 | BENZOQUINONE | 5 | NON- | 5 | TITANIUM | 35 | 1 |
| | POLYOL | | | | YELLOWING | | OXIDE | | |
| | | | | | TYPE | | | | |
| | | | | | XYLYLENE | | | | |
| | | | | | DIISOCYANATE | | | | |
| COMPAR- | EPOXY | 20 | — | — | NON- | 5 | TITANIUM | 35 | 5 |
| ATIVE | POLYOL | | | | YELLOWING | | OXIDE | | |
| EXAMPLE 1 | | | | | TYPE | | | | |
| | | | | | XYLYLENE | | | | |
| | | | | | DIISOCYANATE | | | | |

### Example 1

As shown in Table 1, the coating for medical equipment of Example 1 contains a main agent, a radical scavenger, a curing agent, and a coloring material.

As the main agent, 100 parts by mass of a fluorinated polyol was used. Specifically, F-CLEAR (registered trademark) KD3100 (trade name; manufactured by Kanto Denka Kogyo Co., Ltd.) was used.

As a radical scavenger, 5 parts by mass of hydroquinone was used.

As a curing agent, 24 parts by mass of isocyanate was used. Specifically, TRIXENE BI 7960 (trade name; manufactured by Baxenden Chemicals) was used. TRIXENE BI 7960 is a blocked isocyanate containing dimethylpyrazole (DMP) as a blocking agent.

As a coloring material, 20 parts by weight of titanium oxide (titanium white) was used.

The medical equipment coating of Example 1 was manufactured by mixing each of these components. The medical equipment coating of Example 1 was a one-component reaction type.

In order to form the indicator 2 of Example 1, a coating target member in which a flexible tube 3 in which a stainless steel blade (SUS blade) was spirally wound was coated with an outer-layer resin 4 made of polystyrene resin was manufactured.

The medical equipment coating of Example 1 was applied to the surface of the outer-layer resin 4 of the application target member. The application form of the coating was made to be annular around the outer-layer resin 4.

The application target member to which the medical equipment coating was applied was heated in a heating furnace. As a result, the medical equipment coating was cured, and the indicator 2 of Example 1 was formed on the outer-layer resin 4.

In Example 1, the coat layer 5 was not formed.

The flexible tube 3 of this example and the outer-layer resin 4 on which the indicator 2 was formed were used as test samples for evaluation.

### Example 2

The coating for medical equipment of Example 2 was manufactured in the same manner as the coating for medical equipment of Example 1 except that 5 parts by mass of benzoquinone was used in place of the hydroquinone in Example 1 as a radical scavenger.

The indicator 2 and the test sample in Example 2 were manufactured in the same manner as Example 1 except that the medical equipment coating of Example 2 was used instead of the medical equipment coating of Example 1.

### Example 3

The coating for medical equipment of Example 3 was manufactured in the same way as the coating for medical equipment of Example 1 except that the types of main agent and curing agent and the amount of coloring material were changed and a solvent was used for mixing.

As a main ingredient, in place of the fluorinated polyol in Example 2, 20 parts by mass of epoxy polyol was used. Specifically, EXA-8183 (trade name; manufactured by DIC Corporation) was used as the epoxy polyol.

As a curing agent, 5 parts by mass of non-yellowing xylylene diisocyanate was used in place of the isocyanate in Example 2. Specifically, Takenate (registered trademark) 500 (trade name; manufactured by Mitsui Chemicals, Inc.) was used as the non-yellowing-type xylylene diisocyanate.

The amount of the colorant was 35 parts by weight.

In the medical equipment coating of Example 3, the above-mentioned main agent, radical scavenger, curing agent, and coloring material were mixed together with an organic solvent to produce.

As the organic solvent, a mixed solution of 20 parts by mass of toluene which was an aromatic hydrocarbon, 15 parts by mass of methyl ethyl ketone which was a ketone-based solvent, and 10 parts by mass of isobutyl acetate which was a high boiling point ester-based solvent was used.

The indicator 2 and the test sample in Example 3 were manufactured in the same manner as in Example 2 except that the medical equipment coating of Example 3 was used instead of the medical equipment coating of Example 2. The medical equipment coating of Example 3 was a one-component reaction type.

### Comparative Example 1

The coating for medical equipment of Comparative Example 1 was manufactured in the same manner as the coating for medical equipment of Example 3 except that the radical scavenger was removed from Example 3.

The indicator and the test sample in Comparative Example 1 were manufactured in the same manner as in Example 3 except that the medical equipment coating of Comparative Example 1 was used instead of the medical equipment coating of Example 3.

### (Evaluation)

As shown in Table 1, as the evaluations of Examples 1 to 3 and Comparative Example 1, the “adhesion” evaluation was performed.

In the evaluation of “adhesion”, 200 samples (times) of low-temperature plasma sterilization treatment were performed on each test sample of each example and comparative example 1 using a low-temperature plasma sterilization apparatus. Hydrogen peroxide gas was used as the sterilizing gas in low temperature plasma sterilization.

The adhesion between the indicator and the coat resin in each of the test samples after the above-mentioned sterilization treatment was evaluated using the cross cut method according to JIS K5600-5-6. The evaluation results were represented by the classification 0 to 5 according to the same JIS. The classification number indicates that the smaller the value, the better the adhesion.

As shown in Table 1, the evaluation results of the adhesion in Examples 1 and 2 were all classified into 0. The adhesion between the indicator 2 and the coat resin in Examples 1 and 2 was extremely good.

The evaluation results of adhesion in Example 3 were Class 1. The adhesion in Example 3 was good although slightly inferior to Examples 1 and 2.

The reason why the adhesion of Example 1 or 2 was better than that of Example 3 is considered to be because the fluoro group was introduced into the cured product.

The evaluation result of the adhesion in Comparative Example 1 was Class 5. In Comparative Example 1, it can be seen that the adhesion was significantly reduced after sterilization treatment of 200 cases (times).

The adhesion of the comparative example 1 was significantly reduced as compared with the example 3. In Comparative Example 1, the reason for the decrease in adhesion is considered to be that the coating forming the indicator did not contain a radical scavenger.

As mentioned above, although the preferable embodiment of this invention was described with each Example, this invention is not limited to this embodiment and each Example. Additions, omissions, substitutions, and other modifications of the configuration are possible without departing from the spirit of the present invention.

Further, the present invention is not limited by the above description, and is limited only by the appended claims.

The present invention can be widely applied to coatings for medical equipment and medical equipment, and can improve durability against low temperature plasma sterilization.

What is claimed is:

1. A coating for medical equipment comprising:
an isocyanate-curable coating composition; and
a radical scavenger.

2. The coating for medical equipment according to claim 1, wherein the radical scavenger includes at least one of hydroquinone and benzoquinone.

3. The coating for medical equipment according to claim 1, wherein the isocyanate-curable coating composition includes a fluorine-type compound which introduces a fluoro group into a hardened material after a polymerization reaction.

4. The coating for medical equipment according to claim 1, wherein the isocyanate-curable coating composition includes a main agent and a curing agent for polymerizing the main agent.

5. An endoscope comprising a coating layer formed of a coating for medical equipment, the coating including:
an isocyanate-curable coating composition; and
a radical scavenger.

6. The endoscope according to claim 5, wherein
the endoscope includes an insertion portion inserted into a patient's body,
the insertion portion includes a flexible tubular portion formed in a flexible tube shape,
the flexible tube portion includes a flexible tube and an outer-layer resin covering an outer peripheral portion of the flexible tube in a tubular shape, and
the coating for medical equipment is applied to a surface of the outer-layer resin.
